(19) United States
(12) Patent Application Publication
(10) Pub. No.: US 2024/0115495 A1
(43) Pub. Date: Apr. 11, 2024

(54) FORMULATIONS AND METHODS FOR PULMONARY DELIVERY OF ACTIVE PHARMACEUTICAL INGREDIENTS

(71) Applicant: QNOVIA, INC., RICHMOND, VA (US)

(72) Inventors: Mario Danek, Los Angeles, CA (US); Christopher Kar-Heng Cheng, Los Angeles, CA (US); Joseph Gene Walsh, Los Angeles, CA (US)

(21) Appl. No.: 18/368,042

(22) Filed: Sep. 14, 2023

Related U.S. Application Data
(63) Continuation of application No. 17/681,702, filed on Feb. 25, 2022, now abandoned.
(60) Provisional application No. 63/154,657, filed on Feb. 26, 2021.

(51) Int. Cl. A61K 9/00; A61K 9/107; A61K 9/12; A61K 31/7048; A61K 45/06

(52) U.S. Cl. CPC A61K 9/0073; A61K 9/1075; A61K 9/12; A61K 31/7048; A61K 45/06

(57) ABSTRACT

An aerosolizable liquid formulation for inhalation includes one or more active pharmaceutical ingredients dissolved in a solvent. The one or more active pharmaceutical ingredients includes a vasodilator active pharmaceutical ingredient and, optionally, non-vasodilator active pharmaceutical ingredient.

### CROSS REFERENCE TO RELATED APPLICATIONS

The present application hereby incorporates by reference each of the following U.S. patent applications, as well as any U.S. patent application publication thereof and any U.S. patent issuing therefrom: U.S. patent application Ser. No. 16/657,755, filed Oct. 18, 2019, which '755 application published as U.S. application publication 2020/0060349; U.S. patent application Ser. No. 17/075,679, which '679 application published as U.S. application publication 2021/0113783; and, U.S. patent application Ser. No. 17/518,572. This application further hereby incorporates by reference the disclosure of each patent application from which priority is claimed, any U.S. patent application publications thereof, and any U.S. patents issuing therefrom.

### COPYRIGHT STATEMENT

Any new and original work of authorship in this document is subject to copyright protection under the copyright laws of the United States and other countries. Reproduction by anyone of this document as it appears in official governmental records is permitted, but otherwise all other copyright rights whatsoever are reserved.

### BACKGROUND OF THE INVENTION

The invention generally relates to formulations and methods pertaining to liquids that are aerosolizable and inhalable by persons using electronic devices for the administration and delivery of medicines, pharmaceuticals, vaccines, or treatments to the human body.

Pulmonary delivery is a non-invasive mechanism for local and systemic delivery of therapeutic agents to the human body. Human lungs have a large absorptive surface with high permeability and good blood supply, and absorption of active ingredients can take place quite rapidly. As a result, pulmonary delivery systems continue to feature heavily in current research as interest has grown. In particular, there continues to be research into the types and classes of therapeutics that can be delivered to the human pulmonary system and how those therapeutics can be delivered in a safe and effective manner.

One therapeutic ingredient that has been subject to recent scrutiny is ivermectin, which new research indicates may have a role in treating COVID-19 and/or its associated symptoms. Ivermectin is a well understood and generally safe pharmaceutical ingredient that has existed in tablet form for many years. It is commonly believed to carry a low potential for contraindications. Recent animal studies suggest that ivermectin in a nebulized form may exhibit significantly increased therapeutic efficacy, perhaps even by orders of magnitude greater than other forms. Additionally, in other studies utilizing a cell culture model, ivermectin-loaded nanoparticles (potentially to be delivered orally) have been shown to be effective in reducing cellular uptake of the virus that causes COVID-19.

Accordingly, a need exists for a liquid formulation including one or more active pharmaceutical ingredients that is capable of aerosolization and inhalation by humans for delivery to the pulmonary system safely and effectively. Further a need exists for a method of preparing such a liquid formulation for aerosolization and inhalation by humans for delivery to the pulmonary system. These, and other needs, are believed to be addressed by one or more aspects and features of the invention.

### SUMMARY OF THE INVENTION

The invention includes many aspects and features. Moreover, while many aspects and features relate to, and are described in, the context of formulations and methods for pulmonary delivery of active pharmaceutical ingredients, the invention is not limited to use only in formulations and methods for pulmonary delivery of active pharmaceutical ingredients, as will become apparent from the following summaries and detailed descriptions of aspects, features, and one or more embodiments of the invention.

Broadly defined, the present invention according to one aspect relates to an aerosolizable liquid formulation for inhalation. The formulation comprises one or more active pharmaceutical ingredients dissolved in a solvent. The one or more active pharmaceutical ingredients includes a vasodilator active pharmaceutical ingredient.

In a feature of this aspect, the vasodilator active pharmaceutical ingredient includes one or more of prostacyclin, nitric oxide, milrinone, benazepril, captopril, enalapril, fosinopril, lisinopril, moexipril, perindopril, quinapril, ramipril, trandolapril, azilsartan, candesartan, eprosartan, irbesartan, telmisartan, valsartan, losartan, olmesartan, amlodipine, diltiazem, felodipine, isradipine, nicardipine, nimodipine, nisoldipine, verapamil, nitroglycerin, isosorbide mononitrate, isosorbide dinitrate, hydralazine, minoxidil, fenoldopam, nitroprusside, and alprostadil.

In another feature of this aspect, the solvent includes saline or water.

In another feature of this aspect, the solvent includes one or more of ethanol, vegetable glycerin, and propylene glycol.

In another feature of this aspect, the one or more active pharmaceutical ingredients includes a non-vasodilator active pharmaceutical ingredient.

In another feature of this aspect, the formulation is prepared as a nanoemulsion.

In another feature of this aspect, wherein the non-vasodilator active pharmaceutical ingredient includes ivermectin.

In another feature of this aspect, the non-vasodilator active pharmaceutical ingredient includes a beta-2 agonist active pharmaceutical ingredient. In another feature of this aspect, the beta-2 agonist active pharmaceutical ingredient includes one or more of salbutamol, salmeterol, formoterol, and vilanterol.

In another feature of this aspect, the non-vasodilator active pharmaceutical ingredient includes an anticholinergic active pharmaceutical ingredient. In another feature of this aspect, the anticholinergic active pharmaceutical ingredient includes one or more of ipratropium, tiotropium, aclidinium, glycopyrronium, and theophylline.

In another feature of this aspect, the vasodilator active pharmaceutical ingredient is adapted to increase oxygenation in blood flow upon intake into and delivery to a human pulmonary system.

Broadly defined, the present invention according to another aspect relates to a method of preparing a liquid formulation for aerosolization using an electronic device. The method comprises the steps of: forming an aqueous mixture of water-soluble components and separately forming an oil-based mixture of water-soluble components, wherein at least one of the aqueous mixture and the oil-based mixture includes one or more active pharmaceutical ingredients; adding the oil-based mixture to the aqueous mixture in a homogenizer to form a macroemulsion; and processing the macroemulsion with a microfluidizer to form a nanoemulsion having nanoparticles that include the one or more active pharmaceutical ingredients.

In a feature of this aspect, the method further comprises testing a size of the nanoparticles to ascertain whether the size is within a target range. In another feature of this aspect, the method further comprises processing the nanoemulsion with the microfluidizer one or more additional times if the size is not within the target range. In another feature of this aspect, the target range is approximately 1 micrometer to approximately 3 micrometers. In another feature of this aspect, the target range is approximately less than 5 micrometers.

In another feature of this aspect, the method further comprises loading the nanoemulsion into a cartridge.

In another feature of this aspect, the one or more active pharmaceutical ingredients are dissolved in a solvent that includes one or more of ethanol, vegetable glycerin, and propylene glycol.

In another feature of this aspect, the one or more active pharmaceutical ingredients includes a vasodilator active pharmaceutical ingredient. In another feature of this aspect, the vasodilator active pharmaceutical ingredient includes one or more of prostacyclin, nitric oxide, milrinone, benazepril, captopril, enalapril, fosinopril, lisinopril, moexipril, perindopril, quinapril, ramipril, trandolapril, azilsartan, candesartan, eprosartan, irbesartan, telmisartan, valsartan, losartan, olmesartan, amlodipine, diltiazem, felodipine, isradipine, nicardipine, nimodipine, nisoldipine, verapamil, nitroglycerin, isosorbide mononitrate, isosorbide dinitrate, hydralazine, minoxidil, fenoldopam, nitroprusside, and alprostadil.

In another feature of this aspect, the one or more active pharmaceutical ingredients includes a non-vasodilator active pharmaceutical ingredient. In another feature of this aspect, the non-vasodilator active pharmaceutical ingredient includes ivermectin. In another feature of this aspect, the non-vasodilator active pharmaceutical ingredient includes a beta-2 agonist active pharmaceutical ingredient. In another feature of this aspect, the beta-2 agonist active pharmaceutical ingredient includes one or more of salbutamol, salmeterol, formoterol, and vilanterol. In another feature of this aspect, the non-vasodilator active pharmaceutical ingredient includes an anticholinergic active pharmaceutical ingredient. In another feature of this aspect, the anticholinergic active pharmaceutical ingredient includes one or more of ipratropium, tiotropium, aclidinium, glycopyrronium, and theophylline.

In another feature of this aspect, the one or more active pharmaceutical ingredients includes a vasodilator active pharmaceutical ingredient and a non-vasodilator active pharmaceutical ingredient.

Broadly defined, the present invention according to another aspect relates to a method of administering an aerosolizable and inhalable pharmaceutical complex to a patient. The method comprises the steps of: providing a first cartridge housing a first liquid formulation that includes a vasodilator active pharmaceutical ingredient; providing a second cartridge housing a second liquid formulation that includes a non-vasodilator pharmaceutical ingredient; aerosolizing the first liquid formulation using an electronic device to facilitate inhalation and pulmonary delivery of the vasodilator active pharmaceutical ingredient, thereby increasing oxygenation in a blood flow of the patient; and after increasing oxygenation in the blood flow, aerosolizing the second liquid formulation using the electronic device to facilitate inhalation and pulmonary delivery of the non-vasodilator active pharmaceutical ingredient.

In a feature of this aspect, at least one of the first and second liquid formulations is in an aqueous form.

In another feature of this aspect, at least one of the first and second liquid formulations is in a nanoemulsion form.

Broadly defined, the present invention according to another aspect relates to an aerosolizable liquid formulation for inhalation. The formulation comprises one or more active pharmaceutical ingredients dissolved in a solvent, wherein, upon aerosolization using an electronic device, the one or more active pharmaceutical ingredients exhibit a particle size distribution of less than about 20 micrometers.

In a feature of this aspect, the particle size distribution is less than about 5 micrometers. In another feature of this aspect, the particle size distribution is between about 1 micrometer and about 3 micrometers. In another feature of this aspect, an aerosolized form of the liquid formulation exhibits a generally laminar flow.

In a feature of this aspect, the particle size distribution is greater than about 5 micrometers. In another feature of this aspect, the particle size distribution is between about 5 micrometers and about 20 micrometers. In another feature of this aspect, an aerosolized form of the liquid formulation exhibits a generally turbulent flow.

In another feature of this aspect, the one or more active pharmaceutical ingredients includes povidone-iodine. In another feature of this aspect, the solvent includes saline or water.

Broadly defined, the present invention according to another aspect relates to a method of administering an aerosolizable and inhalable pharmaceutical complex to a patient. The method comprises the steps of: providing a first cartridge housing a first liquid formulation that includes an active pharmaceutical ingredient that exhibits a first particle size distribution; providing a second cartridge housing a second liquid formulation that includes an active pharmaceutical ingredient that exhibits a second particle size distribution different than the first particle size distribution; aerosolizing the first liquid formulation using an electronic device; and after aerosolizing the first liquid formulation, aerosolizing the second liquid formulation.

In a feature of this aspect, the active pharmaceutical ingredient of the first liquid formulation and the active pharmaceutical ingredient of the second liquid formulation include a common active pharmaceutical ingredient. In another feature of this aspect, the common active pharmaceutical ingredient includes povidone-iodine. In another feature of this aspect, the first liquid formulation exhibits a generally turbulent flow upon aerosolization. In another feature of this aspect, the second liquid formulation exhibits a generally laminar flow upon aerosolization.

Broadly defined, the present invention according to another aspect relates to a liquid formulation for preparation as part of an aerosolizable complex for inhalation substantially as shown and described.

Broadly defined, the present invention according to another aspect relates to a liquid formulation for aerosolization including one or more active pharmaceutical ingredients, substantially as shown and described.

Broadly defined, the present invention according to another aspect relates to a method of preparing a liquid formulation for aerosolization using an electronic device substantially as shown and described.

Broadly defined, the present invention according to another aspect relates to a method of administering an aerosolizable and inhalable pharmaceutical complex to a patient substantially as shown and described.

In addition to the aforementioned aspects and features of the invention, it should be noted that the invention further encompasses the various logical combinations and subcombinations of such aspects and features. Thus, for example, claims in this or a divisional or continuing patent application or applications may be separately directed to any aspect, feature, or embodiment disclosed herein, or combination thereof, without requiring any other aspect, feature, or embodiment.

### BRIEF DESCRIPTION OF THE DRAWINGS

One or more preferred embodiments of the invention now will be described in detail with reference to the accompanying drawings, wherein the same elements are referred to with the same reference numerals, and wherein,

FIG. 1 is a flowchart illustrating a method of administering a sequence of cartridges, each of which includes an active pharmaceutical ingredient, in accordance with one or more aspects of the present invention;

FIG. 2 is a flowchart illustrating a method of administering a sequence of cartridges, each of which includes an active pharmaceutical ingredient that exhibits a different particle size distribution, in accordance with one or more aspects of the present invention; and

FIG. 3 is a flowchart illustrating a method of preparing a nanoemulsion that includes an active pharmaceutical ingredient in accordance with one or more aspects of the present invention.

### DETAILED DESCRIPTION

As a preliminary matter, it will readily be understood by one having ordinary skill in the relevant art (“Ordinary Artisan”) that the invention has broad utility and application. Furthermore, any embodiment discussed and identified as being “preferred” is considered to be part of a best mode contemplated for carrying out the invention. Other embodiments also may be discussed for additional illustrative purposes in providing a full and enabling disclosure of the invention. Furthermore, an embodiment of the invention may incorporate only one or a plurality of the aspects of the invention disclosed herein; only one or a plurality of the features disclosed herein; or combination thereof. As such, many embodiments are implicitly disclosed herein and fall within the scope of what is regarded as the invention.

Accordingly, while the invention is described herein in detail in relation to one or more embodiments, it is to be understood that this disclosure is illustrative and exemplary of the invention and is made merely for the purposes of providing a full and enabling disclosure of the invention. The detailed disclosure herein of one or more embodiments is not intended, nor is to be construed, to limit the scope of patent protection afforded the invention in any claim of a patent issuing here from, which scope is to be defined by the claims and the equivalents thereof. It is not intended that the scope of patent protection afforded the invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the invention. Accordingly, it is intended that the scope of patent protection afforded the invention be defined by the issued claim(s) rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

With regard to any method claim including a condition precedent step, such method requires the condition precedent to be met and the step to be performed at least once but not necessarily every time during performance of the claimed method.

Furthermore, it is important to note that, as used herein, “comprising” is open-ended insofar as that which follows such term is not exclusive. Additionally, “a” and “an” each generally denotes “at least one” but does not exclude a plurality unless the contextual use dictates otherwise. Thus, reference to “a picnic basket having an apple” is the same as “a picnic basket comprising an apple” and “a picnic basket including an apple”, each of which identically describes “a picnic basket having at least one apple” as well as “a picnic basket having apples”; the picnic basket further may contain one or more other items beside an apple. In contrast, reference to “a picnic basket having a single apple” describes “a picnic basket having only one apple”; the picnic basket further may contain one or more other items beside an apple. In contrast, “a picnic basket consisting of an apple” has only a single item contained therein, i.e., one apple; the picnic basket contains no other item.

When used herein to join a list of items, “or” denotes “at least one of the items” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers”; the picnic basket further may contain one or more other items beside cheese and crackers.

When used herein to join a list of items, “and” denotes “all of the items of the list”. Thus, reference to “a picnic basket having cheese and crackers” describes “a picnic basket having cheese, wherein the picnic basket further has crackers”, as well as describes “a picnic basket having crackers, wherein the picnic basket further has cheese”; the picnic basket further may contain one or more other items beside cheese and crackers.

The phrase “at least one” followed by a list of items joined by “and” denotes an item of the list but does not require every item of the list. Thus, “at least one of an apple and an orange” encompasses the following mutually exclusive scenarios: there is an apple but no orange; there is an orange but no apple; and there is both an apple and an orange. In these scenarios if there is an apple, there may be more than one apple, and if there is an orange, there may be more than one orange. Moreover, the phrase “one or more” followed by a list of items joined by “and” is the equivalent of “at least one” followed by the list of items joined by “and”.

Referring now to the drawings, one or more preferred embodiments in accordance with one or more aspects and features of the invention are next described. The following description of one or more preferred embodiments is merely exemplary in nature and is in no way intended to limit the invention, its implementations, or uses.

Liquid Formulations. A liquid formulation capable of aerosolization and inhalation by humans for delivery to the pulmonary system in accordance with one or more aspects of the present invention includes one or more active pharmaceutical ingredients mixed with a solvent. It is further contemplated that a liquid formulation capable of aerosolization and inhalation by humans for delivery to the pulmonary system may exist in an aqueous form or as an emulsion or nanoemulsion. In this regard, it is contemplated that a solvent may be selected to help solubilize the one or more active pharmaceutical ingredients. In at least some contemplated embodiments, the one or more active pharmaceutical ingredients includes at least one vasodilator active pharmaceutical ingredient and at least one non-vasodilator active pharmaceutical ingredient.

It is contemplated that any of a wide range of different active pharmaceutical ingredients are capable of use in connection with a liquid formulation having at least one vasodilator active pharmaceutical ingredient and at least one non-vasodilator active pharmaceutical ingredient. In at least some contemplated embodiments, a liquid formulation in accordance with one or more aspects of the present invention includes ivermectin as a non-vasodilator active pharmaceutical ingredient. Ivermectin is understood to have potential for treating COVID-19 and/or its associated symptoms, and within the context of targeted delivery to the pulmonary system, may exhibit greatly enhanced treatment efficacy as compared with systemic delivery. Other contemplated active pharmaceutical ingredients for use in connection with a liquid formulation in accordance with one or more aspects of the present invention are identified hereinbelow. Additionally, in at least some embodiments, a liquid formulation in accordance with one or more aspects of the present invention includes one or more of the following vasodilator active pharmaceutical ingredients: prostacyclin; nitric oxide; and milrinone.

It is contemplated that any of a wide variety of different solvents may be used with a liquid formulation in accordance with one or more aspects of the present invention. Contemplated solvents include, but are not limited to, one or more of ethanol, vegetable glycerin, and propylene glycol. Furthermore, it is contemplated that one or more solvents may be selected to help solubilize specific selected active pharmaceutical ingredients.

It is contemplated that use of a vasodilator active pharmaceutical ingredient together with a non-vasodilator active pharmaceutical ingredient can increase oxygenation in blood flow. Pulmonary vasodilators are understood to improve gas exchange and heart functionality by increasing the level of oxygen carried in the blood flow. A vasodilator active pharmaceutical ingredient that is inhaled would be delivered to well-ventilated regions of the lung, which is understood to result in preferentially directing higher levels of oxygenation at pulmonary vessels in such well-ventilated regions.

Inclusion of a vasodilator active pharmaceutical ingredient in a liquid formulation in accordance with one or more aspects of the present invention can increase oxygenation for enhanced and/or effective delivery of another active pharmaceutical ingredient within the human body. Additionally, inclusion of a vasodilator active pharmaceutical ingredient in a liquid formulation in accordance with one or more aspects of the present invention can increase oxygenation as a preferred outcome in certain therapy regimens.

It is contemplated that a vasodilator active pharmaceutical ingredient may be selected on the basis of half-life. In particular, a shorter half-life for the vasodilator active pharmaceutical ingredient may reduce the likelihood of recirculation so that a focus is on pulmonary circulation.

In addition to at least one vasodilator active pharmaceutical ingredient, at least one non-vasodilator active pharmaceutical ingredient, and a solvent, it is further contemplated that a liquid formulation in accordance with one or more aspects of the present invention may also include one or more of the following: water; a saline; one or more surfactants; one or more acids; one or more bases; and one or more preservatives. Liquid formulations that include one or more of the foregoing ingredients, including methods of preparing such formulations, are disclosed in the '679 Application.

A liquid formulation having at least one vasodilator active pharmaceutical ingredient, at least one non-vasodilator active pharmaceutical ingredient, a solvent, and one or more other ingredients described above can be prepared in accordance with methods described hereinbelow and placed in a single cartridge for storage and later administration using an electronic device for aerosolizing the liquid formulation to be inhaled. Contemplated electronic devices are described in the incorporated applications, patents, and patent application publications, including the '679 application, the '881 application, and the '755 application (collectively the “Incorporated Disclosures”).

Active pharmaceutical ingredients may be deliverable to the human body (and, in particular, to the pulmonary system) in a variety of different ways. In various contemplated embodiments, one or more active pharmaceutical ingredients are capable of delivery using electronic devices (such as those described in the Incorporated Disclosures) to the nasal region, to the buccal region, to the upper lung region, to the alveolar lung region, and/or to the deep lung region. When a liquid formulation that includes one or more active pharmaceutical ingredients is aerosolized, the particle size distribution, or a mass median aerodynamic diameter (MMAD), of the one or more active pharmaceutical ingredients may direct or enhance delivery and intake of the one or more active pharmaceutical ingredients.

In some contemplated embodiments, an aerosolized form of the liquid formulation including one or more active pharmaceutical ingredients embodies a generally laminar flow, whereby a particle size distribution of the aerosolized active pharmaceutical ingredient(s) is less than about 5 micrometers. In a preferred embodiment, the aerosolized form has a particle size distribution that is between about 1 micrometer to about 3 micrometers. With a particle size distribution that imparts the aerosolized form with a generally laminar flow, the one or more active pharmaceutical ingredients may be deliverable to the deep lung region, where the one or more active pharmaceutical ingredients may undergo alveolar deposition. In this regard, the one or more active pharmaceutical ingredients can be targeted not only to the respiratory tract, but also throughout the body via the bloodstream.

In other contemplated embodiments, an aerosolized form of the liquid formulation including one or more active pharmaceutical ingredients embodies a more turbulent flow. Here, it is contemplated that a particle size distribution of the aerosolized active pharmaceutical ingredient(s) is greater than about 5 micrometers, and preferably between about 5 micrometers and about 20 micrometers. This slightly larger particle size, with wider distribution in particle size, can impart the aerosolized form of the liquid formulation with a wetting, mist-like deposition. With a mist-like deposition, the one or more active pharmaceutical ingredients may be deliverable to the human body by nasal or buccal (oral) administration.

In accordance with the foregoing discussion, it is contemplated that a particle size distribution of the one or more active pharmaceutical ingredients may be useful in determining areas of targeted delivery within the human body and may further be useful in determining whether the one or more active pharmaceutical ingredients operate as a treatment of existing symptoms or as a prophylactic treatment. One contemplated instance of this paradigm involves an active pharmaceutical ingredient that includes povidone-iodine, which is another active pharmaceutical ingredient that is understood to have potential for treating COVID-19 and/or its associated symptoms and may also have use as a prophylactic treatment for COVID-19.

It is contemplated that povidone-iodine can be dissolved in a saline solvent (e.g., NaCl solution) and placed in a cartridge for storage and later administration using an electronic device for aerosolizing the liquid formulation.

In cases where povidone-iodine in the cartridge exhibits a particle size distribution of less than about 5 micrometers, and preferably between about 1 micrometer and 3 micrometers, an aerosolized form of the liquid formulation may exhibit a generally laminar flow. In this regard, povidone-iodine may be deliverable to the deep lung region, where it may undergo alveolar deposition and be absorbed into the bloodstream. As such, with a particle size distribution of less than about 5 micrometers, and preferably between about 1 micrometer and 3 micrometers, povidone-iodine may be used for prophylactic targeting of the respiratory tract as well as the body systemically via the bloodstream.

In cases where povidone-iodine in the cartridge exhibits a particle size distribution of greater than about 5 micrometers, and preferably between about 5 micrometers and 20 micrometers, an aerosolized form of the liquid formulation may exhibit a more turbulent flow, which can impart the aerosolized form with a wetting, mist-like deposition. In this regard, povidone-iodine may be deliverable to the human body by nasal or buccal (oral) administration, thereby targeting an area (the nasal cavity and buccal area) where SARS-CoV-2 often first enters the human body and replicates to create an infection that ultimately spreads to other systems. As such, with a particle size distribution of greater than about 5 micrometers, and preferably between about 5 micrometers and 20 micrometers, povidone-iodine may be used for prophylactic targeting of the nasal cavity and buccal area where the virus that causes a COVID-19 infection is often first received and incubated.

In at least some embodiments, it may be advantageous to prepare separate liquid formulations in accordance with one or more aspects of the present invention. In such cases, it is contemplated that one liquid formulation may include a vasodilator active pharmaceutical ingredient in a solvent, and a separate liquid formulation may include a non-vasodilator active pharmaceutical ingredient in a solvent. Each separate liquid formulation may also include one or more of the following: water; a saline; one or more surfactants; one or more acids; one or more bases; and one or more preservatives. Additionally, each separate liquid formulation may be prepared in the manner described in the '679 Application. In this regard, each separate liquid formulation can be placed in a separate cartridge, and each cartridge can be administered separately using an electronic device as described in each of the Incorporated Disclosures.

In a similar manner, it may be advantageous to prepare separate liquid formulations in accordance with one or more aspects of the present invention, where the same one or more active pharmaceutical ingredients are used in each separate liquid formulation, but at different particle size distributions. Each separate liquid formulation may be prepared in the manner described in the '679 Application. In this regard, each separate liquid formulation can be placed in a separate cartridge, and each cartridge can be administered separately using an electronic device as described in each of the Incorporated Disclosures. Such an approach may be useful, for example, in the administration and delivery of povidone-iodine as a prophylactic or treatment for COVID-19 or symptoms associated therewith. One cartridge may contain a liquid formulation that includes a saline solvent with povidone-iodine having a particle size distribution of less than about 5 micrometers, and preferably between about 1 micrometer and 3 micrometers. Another separate cartridge may contain a liquid formulation that includes a saline solvent with povidone-iodine having a particle size distribution of greater than about 5 micrometers, and preferably between about 5 micrometers and 20 micrometers.

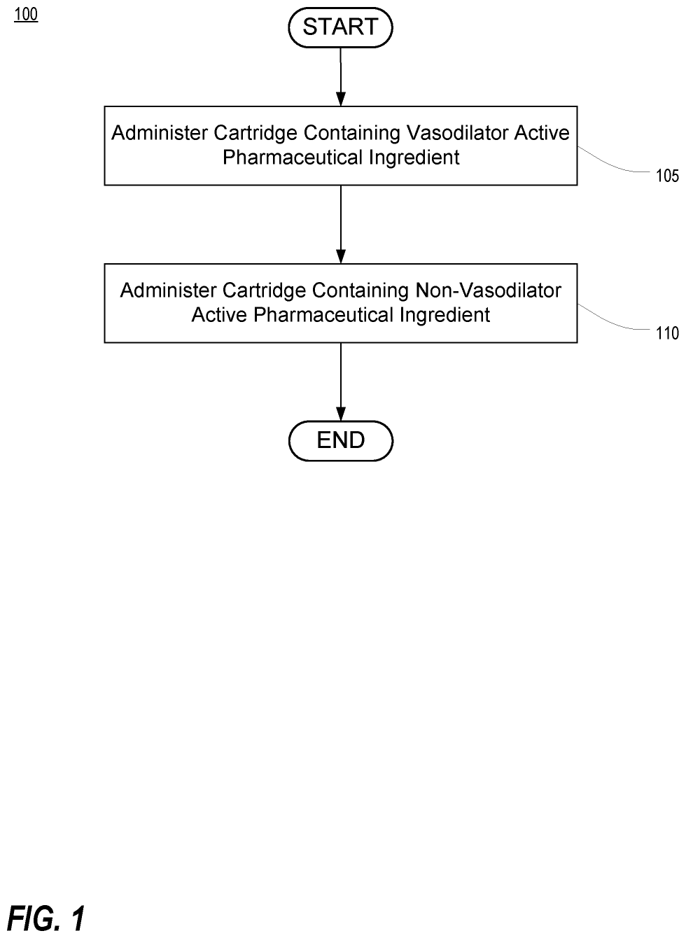

Method of Administering Liquid Formulations Sequentially. FIG. 1 is a flowchart illustrating a method 100 of administering a sequence of cartridges, each of which includes an active pharmaceutical ingredient, in accordance with one or more aspects of the present invention. As shown in FIG. 1, it is contemplated that a cartridge containing a vasodilator active pharmaceutical ingredient is administered at step 105 using an electronic device for aerosolizing the cartridge contents prior to administration of a cartridge containing a non-vasodilator active pharmaceutical ingredient at step 110. In this regard, the vasodilator active pharmaceutical ingredient can be aerosolized and inhaled for delivery to the pulmonary system first so as to increase oxygenation in the blood flow. Increased oxygenation may itself be a preferred outcome of a therapy regimen, or increased oxygenation may enhance the intake and delivery of a non-vasodilator active pharmaceutical ingredient contained within a subsequently-administered cartridge.

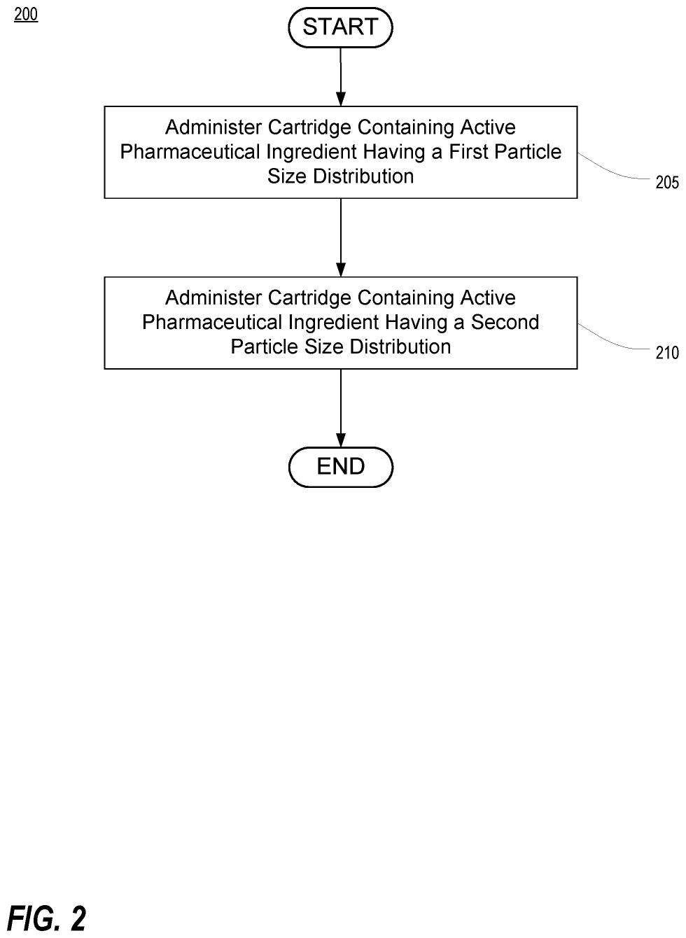

FIG. 2 is a flowchart illustrating a method 200 of administering a sequence of cartridges, each of which includes an active pharmaceutical ingredient that exhibits a different particle size distribution, in accordance with one or more aspects of the present invention. As shown in FIG. 2, it is contemplated that cartridges that include active pharmaceutical ingredients with different particle size distributions can be administered sequentially at steps 205 and 210. In contemplated embodiments, a selected active pharmaceutical ingredient for each cartridge may be the same, or they may be different, to satisfy treatment objectives.

It is further contemplated that the method of sequential administration of FIG. 2 may be used to administer cartridges that each carry a liquid formulation that includes povidone-iodine, but at different particle size distributions. In this regard, it is contemplated that a cartridge that includes povidone-iodine at a particle size distribution to support a turbulent aerosolized form (and a wetting, mist-like deposition) can be administered at step 205 to provide prophylactic treatment in response to a possible COVID-19 infection in the nasal cavity or buccal area. Then, at step 210, a cartridge that includes povidone-iodine at a particle size distribution to support a generally laminar aerosolized form can be administered to provide prophylactic treatment to the respiratory tract and to provide targeted delivery for alveolar deposition.

Returning to the discussion in connection with FIG. 1, a liquid formulation in accordance with one or more aspects of the present invention that includes at least one vasodilator active pharmaceutical ingredient (but not a non-vasodilator active pharmaceutical ingredient) may include any of a wide range of different vasodilator active pharmaceutical ingredients. In at least some embodiments, such a liquid formulation includes one or more of the following vasodilator active pharmaceutical ingredients: prostacyclin; nitric oxide; and milrinone.

Additionally, a liquid formulation in accordance with one or more aspects of the present invention that includes at least one non-vasodilator active pharmaceutical ingredient (but not a vasodilator active pharmaceutical ingredient) may include any of a wide range of different non-vasodilator active pharmaceutical ingredients. In at least some embodiments, such a liquid formulation includes ivermectin as an active pharmaceutical ingredient. In other contemplated embodiments, such a liquid formulation includes one or more beta-2 agonist active pharmaceutical ingredients. Contemplated beta-2 agonist active pharmaceutical ingredients include, but are not limited to: salbutamol; salmeterol; formoterol; and vilanterol. In still other contemplated embodiments, such a liquid formulation includes one or more anticholinergic active pharmaceutical ingredients. Contemplated anticholinergic active pharmaceutical ingredients include, but are not limited to: ipratropium; tiotropium; aclidinium; glycopyrronium; and theophylline. Still other contemplated active pharmaceutical ingredients for use in connection with a liquid formulation in accordance with one or more aspects of the present invention are identified hereinbelow.

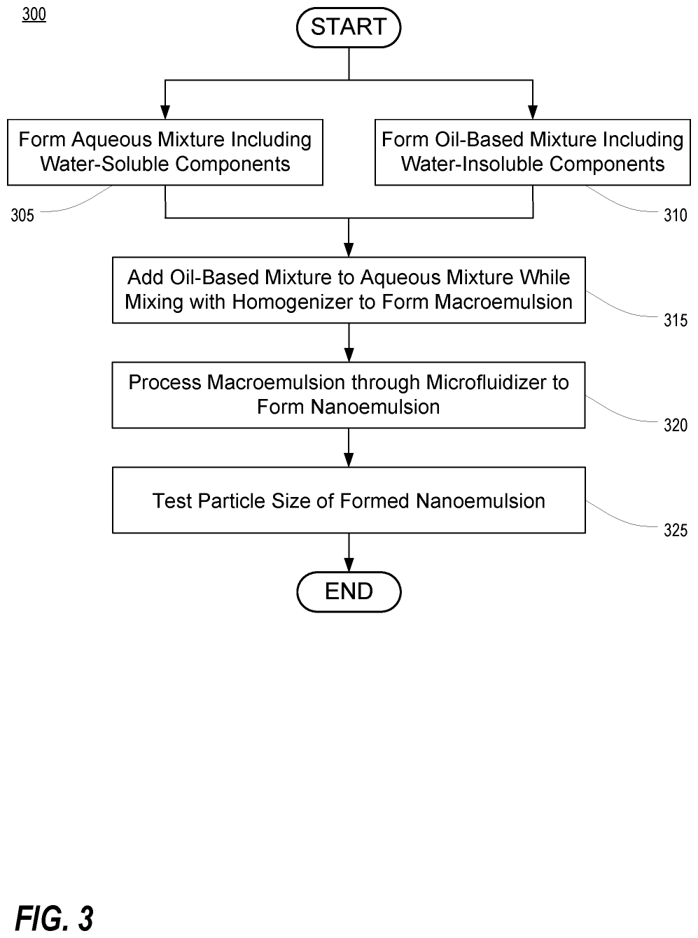

Some liquid formulations in accordance with one or more aspects of the present invention can be prepared as a nanoemulsion for packaging in a cartridge for storage and later administration to the pulmonary system, while other liquid formulations in accordance with one or more aspects of the present invention may exist in an aqueous form. Preparation as a nanoemulsion can be undertaken irrespective of whether the liquid formulation includes a vasodilator active pharmaceutical ingredient, a non-vasodilator active pharmaceutical ingredient, or each of a vasodilator and non-vasodilator active pharmaceutical ingredient. In this regard, FIG. 3 is a flowchart illustrating a method 300 of preparing a nanoemulsion that includes an active pharmaceutical ingredient in accordance with one or more aspects of the present invention.

As discussed above, a liquid formulation in accordance with one or more aspects of the present invention includes an active pharmaceutical ingredient (whether a vasodilator, a non-vasodilator, or one or more of each), a solvent, and one or more of the following: water; a saline; one or more surfactants; one or more acids; one or more bases; and one or more preservatives. It is contemplated that one or more solvents may be selected to help solubilize specific selected active pharmaceutical ingredients, which can also promote stability in the resulting nanoemulsion. Although it is contemplated that any of a wide variety of different solvents may be used with a liquid formulation in accordance with one or more aspects of the present invention, some contemplated solvents include, but are not limited to, one or more of ethanol, vegetable glycerin, and propylene glycol.

Method of Preparing Liquid Formulation as a Nanoemulsion. For some liquid formulations in accordance with one or more aspects of the present invention, it may be preferable to prepare the liquid formulation as a nanoemulsion prior to loading the formulation into a cartridge. A method 300 for preparing a nanoemulsion involves creating an aqueous mixture made from water-soluble ingredients of the liquid formulation at step 305. Separately, at step 310, an oil-based mixture is made of water-insoluble ingredients of the liquid formulation. The aqueous mixture and the oil-based mixture are combined and mixed together at step 315 to create a macroemulsion. In particular, it is contemplated that the oil-based mixture, which generally occupies less total volume than the aqueous mixture, is gradually added to the aqueous mixture while mixing with a homogenizer.

Following the formation of the macroemulsion, the combined mixture can be processed with one or more passes through a microfluidizer at step 320 to form a nanoemulsion. In at least some embodiments, the combined mixture is passed through the microfluidizer approximately three to five times. A microfluidizer is a machine that uses microreactor technology to form nanoemulsions through the interaction of liquid streams in defined microchannels. Advantageously, a microfluidizer is capable of creating nanoemulsions from a wide variety of different oil-inclusive mixtures. The network of channels of the microfluidizer includes very small dimensions to process the mixture in a manner to achieve a homogeneity with consistently-sized nanoparticles. Further details of a microfluidics technique of forming a nanoemulsion can be found in the '679 Application.

Additionally, microfluidizer technology is described, for example, in U.S. Patent Application Publication Nos. US 2012/0236680 and US 2019/0299171, each of which is incorporated herein by reference. Preferred microfluidizing machines correspond to the processors manufactured, sold, or distributed by Mircofluidics of Newton or Westwood, Massachusetts, under the registered trademark MICROFLUIDIZER, and any and all other apparatus that have the same or equivalent structure for performing the same or equivalent function with the same or equivalent result. Appendix A hereof includes a user guide from 2014 for MICROFLUIDIZER processors distributed by Microfluidics, which Appendix A is incorporated herein by reference.

After passing the mixture through the microfluidizer, testing for appropriately sized particles can be accomplished at step 325. The resulting nanoemulsion should exhibit particle size smaller than 5 micrometers, with a target of approximately 1 micrometer to approximately 3 micrometers for enhanced stability of the nanoemulsion. If testing does not yield results within the preferred target range, the mixture can be passed through the microfluidizer again.

As compared with other techniques for creating a nanoemulsion, a microfluidics approach has the benefit of producing nanoemulsions that require lower amounts of surfactant(s), with a lower chance of contamination with process excipients, which is conducive to pharmaceutical applications in terms of meeting toxicological and human clinical safety standards. Additionally, a microfluidics approach produces highly stable nanoemulsions with better particle size distribution. When loaded into a cartridge, the nanoemulsion is also provided with the benefit of longer shelf life owing in part to its enhanced stability.

It is contemplated that nanoparticles of the resulting nanoemulsion may take the form of micelles or liposomes that include the one or more active pharmaceutical ingredients for delivery to the human pulmonary system. Further discussion of nanoparticle structure can be found in the '679 Application. When the nanoemulsion is loaded into a cartridge, an electronic device such as those described in each of the Incorporated Disclosures can be used to aerosolize the nanoemulsion so that the one or more active pharmaceutical ingredients can be inhaled and delivered to the pulmonary system.

Examples. In the discussion that follows, a variety of different indications, which may be treated or otherwise addressed by one or more active pharmaceutical ingredients capable of inclusion in a liquid formulation in accordance with one or more aspects of the present invention, are disclosed. For each indication, a nonexclusive list of contemplated active pharmaceutical ingredients, which may be used to treat or otherwise address each identified indication upon delivery to the human pulmonary system, are likewise disclosed.

It is contemplated that some of the active pharmaceutical ingredients identified in the foregoing discussion as well as hereinbelow may solubilize in an aqueous solution, which mixture may optionally be mixed with one or more other components and packaged into a cartridge for aerosolization. It is further contemplated that some of the active pharmaceutical ingredients identified in the foregoing discussion as well as hereinbelow may be prepared as part of a nanoemulsion using a microfluidics approach as discussed hereinabove in connection with FIG. 3.

Additionally, it is contemplated that active pharmaceutical ingredients identified in the foregoing discussion as well as hereinbelow may be deliverable to the human body (and, in particular, to the pulmonary system) in a variety of different ways. For example, some contemplated active pharmaceutical ingredients capable of inclusion in a liquid formulation in accordance with one or more aspects of the present invention may be delivered nasally. Some contemplated active pharmaceutical ingredients capable of inclusion in a liquid formulation in accordance with one or more aspects of the present invention may be delivered to the buccal region. Additionally, some contemplated active pharmaceutical ingredients capable of inclusion in a liquid formulation in accordance with one or more aspects of the present invention may be delivered to the lungs, such as the upper lung region, the deep lung region, and/or the alveolar lung region.

In some contemplated embodiments, an active pharmaceutical ingredient may be used in the treatment of anaphylaxis. Contemplated active pharmaceutical ingredients in this regard include, but are not limited to, epinephrine.

In other contemplated embodiments, an active pharmaceutical ingredient may include a sedative or premedicant for induction of anesthesia. Contemplated anesthetics in this regard include, but are not limited to: desflurane; midazolam; and oxymetazoline hydrochloride+tetracaine hydrochloride combination.

In other contemplated embodiments, an active pharmaceutical ingredient may be used in the treatment of aspergillus infection or associated symptoms. Contemplated active pharmaceutical ingredients in this regard include, but are not limited to, caspofungin acetate.

In other contemplated embodiments, an active pharmaceutical ingredient may be used in the treatment of asthma or associated symptoms. Contemplated active pharmaceutical ingredients in this regard include, but are not limited to: acetylcysteine; albuterol sulfate; arformoterol tartrate; azelastine hydrochloride; beclometasone dipropionate; beclometasone dipropionate hydrofluoroalkane; beclometasone+formoterol combination; beclometasone+salbutamol combination; budesonide; budesonide hydrofluoroalkane; budesonide+formoterol combination; ciclesonide; cromolyn sodium; epinephrine racemic; fluticasone; filiticasonc+saimetcrol combination: fluticasone propionate+formoterol fumarate dihydrate combination; formoterol fumarate; formoterol hydrofluoroalkane; formoterol+fluticasone propionate combination; ipratropium bromide+albuterol sulfate combination; levalbuterol; mometasone furoate; nedocromil; olodaterol; pirbuterol; procaterol; salbutamol; (R)-salbutamol; salbutamol+ipratropium bromide combination; salmeterol; and triamcinolone acetonide.

In other contemplated embodiments, an active pharmaceutical ingredient may be used in the treatment of bronchiectasis or associated symptoms. Contemplated active pharmaceutical ingredients in this regard include, but are not limited to: aztreonam lysine; beclometasone+formoterol combination; colistimethate sodium; levofloxacin; and molgramostim.

In other contemplated embodiments, an active pharmaceutical ingredient may be used in the treatment of bronchiolitis obliterans or associated symptoms. Contemplated active pharmaceutical ingredients in this regard include, but are not limited to, ciclosporin A.

In other contemplated embodiments, an active pharmaceutical ingredient may be used in the treatment of candidiasis infection or associated symptoms. Contemplated active pharmaceutical ingredients in this regard include, but are not limited to, caspofungin acetate.

In other contemplated embodiments, an active pharmaceutical ingredient may be used in the treatment of chronic bronchitis or associated symptoms. Contemplated active pharmaceutical ingredients in this regard include, but are not limited to: arformoterol tartrate; and pirbuterol.

In other contemplated embodiments, an active pharmaceutical ingredient may be used in the treatment of chronic obstructive pulmonary disease (COPD) or associated symptoms. Contemplated active pharmaceutical ingredients in this regard include, but are not limited to: albuterol sulfate; arformoterol; arformoterol tartrate; beclometasone+formoterol combination; budesonide; budesonide+formoterol combination; ciclesonide; formoterol; formoterol fumarate; formoterol hydrofluoroalkane; formoterol+fluticasone propionate combination; glycopyrrolate; ipratropium; ipratropium bromide; ipratropium bromide+albuterol sulfate combination; levalbuterol; levofloxacin; olodaterol; olodaterol+tiotropium bromide combination; recombinant deoxyribonuclease; (R)-salbutamol; and salbutamol+ipratropium bromide combination.

In other contemplated embodiments, an active pharmaceutical ingredient may be used in the treatment or prevention of coronavirus infection (such as infection arising from SARS-CoV-2) or associated symptoms. Contemplated active pharmaceutical ingredients in this regard include, but are not limited to: ciclesonide; ivermectin, povidone-iodine, and remdesivir.

In other contemplated embodiments, an active pharmaceutical ingredient may be used in the treatment of croup or associated symptoms. Contemplated active pharmaceutical ingredients in this regard include, but are not limited to, budesonide.

In other contemplated embodiments, an active pharmaceutical ingredient may be used in the treatment of cystic fibrosis or associated symptoms. Contemplated active pharmaceutical ingredients in this regard include, but are not limited to: aztreonam lysine; dornase alfa; hypertonic sodium chloride; hypotonic sodium chloride; isotonic sodium chloride; levofloxacin; lyophilized preparation of highly purified human alpha-1 protease inhibitor; molgramostim; recombinant deoxyribonuclease; and tobramycin.

In other contemplated embodiments, an active pharmaceutical ingredient may be used in the treatment of cytokine release syndrome or associated symptoms. Contemplated active pharmaceutical ingredients in this regard include, but are not limited to, amnion-derived cellular cytokine solution.

In other contemplated embodiments, an active pharmaceutical ingredient may include a medication for treatment of drug poisoning Contemplated medications for treatment of pain in this regard include, but are not limited to: naloxone; and naloxone hydrochloride.

In other contemplated embodiments, an active pharmaceutical ingredient may be used in the treatment of emphysema or associated symptoms. Contemplated active pharmaceutical ingredients in this regard include, but are not limited to: amnion-derived cellular cytokine solution; arformoterol tartrate; lyophilized preparation of highly purified human alpha-1 protease inhibitor; and pirbuterol.

In other contemplated embodiments, an active pharmaceutical ingredient may be used in the treatment of a mycobacterial infection or associated symptoms. Contemplated active pharmaceutical ingredients in this regard include, but are not limited to: levofloxacin; and molgramostim.

In other contemplated embodiments, an active pharmaceutical ingredient may include a medication for treatment of narcotics addiction. Contemplated medications for treatment of pain in this regard include, but are not limited to, naloxone hydrochloride.

In other contemplated embodiments, an active pharmaceutical ingredient may be used in the treatment of nasal polyps or associated symptoms. Contemplated active pharmaceutical ingredients in this regard include, but are not limited to: budesonide; fluticasone propionate; and mometasone furoate.

In other contemplated embodiments, an active pharmaceutical ingredient may include a medication for treatment of pain and/or pain management. Contemplated medications for treatment of pain in this regard include, but are not limited to: fentanyl; fentanyl citrate; and ketorolac.

In other contemplated embodiments, an active pharmaceutical ingredient may be used as a prophylactic for or in the treatment of pneumocystis jirovecii. Contemplated active pharmaceutical ingredients in this regard include, but are not limited to: caspofungin acetate; and pentamidine.

In other contemplated embodiments, an active pharmaceutical ingredient may be used in the treatment of a pseudomonal infection or associated symptoms. Contemplated active pharmaceutical ingredients in this regard include, but are not limited to: aztreonam lysine; colistimethate sodium; levofloxacin; and tobramycin.

In other contemplated embodiments, an active pharmaceutical ingredient may be used in the treatment of pulmonary alveolar proteinosis or associated symptoms. Contemplated active pharmaceutical ingredients in this regard include, but are not limited to, molgramostim.

In other contemplated embodiments, an active pharmaceutical ingredient may be used in the treatment of pneumocystis pneumonia or associated symptoms. Contemplated active pharmaceutical ingredients in this regard include, but are not limited to, pentamidine.

In other contemplated embodiments, an active pharmaceutical ingredient may be used in the treatment of pulmonary hypertension or associated symptoms. Contemplated active pharmaceutical ingredients in this regard include, but are not limited to: iloprost; iloprost trometamol; and treprostinil.

In other contemplated embodiments, an active pharmaceutical ingredient may be used in the treatment of pulmonary inflammation or associated symptoms. Contemplated active pharmaceutical ingredients in this regard include, but are not limited to, vazegepant.

In other contemplated embodiments, an active pharmaceutical ingredient may be used in the treatment of respiratory distress syndrome or associated symptoms. Contemplated active pharmaceutical ingredients in this regard include, but are not limited to: molgramostim; and poractant alfa.

In other contemplated embodiments, an active pharmaceutical ingredient may be used in the treatment of a respiratory tract infection or associated symptoms. Contemplated active pharmaceutical ingredients in this regard include, but are not limited to: aztreonam lysine; immunostimulant ribosomal vaccine; and tobramycin.

In other contemplated embodiments, an active pharmaceutical ingredient may be used in the treatment of rhinitis or associated symptoms. Contemplated active pharmaceutical ingredients in this regard include, but are not limited to: azelastine hydrochloride; beclometasone dipropionate; budesonide; ciclesonide; fluticasone furoate; fluticasone propionate; interferon gamma; mometasone furoate; nedocromil; olopatadine; olopatadine hydrochloride+mometasone furoate combination; triamcinolone acetonide; spaglumic acid; and xylometazoline.

In other contemplated embodiments, an active pharmaceutical ingredient may be used in the treatment of rhinovirus infection or associated symptoms. Contemplated active pharmaceutical ingredients in this regard include, but are not limited to, povidone-iodine.

In other contemplated embodiments, an active pharmaceutical ingredient may be used in the treatment of sinusitis or associated symptoms. Contemplated active pharmaceutical ingredients in this regard include, but are not limited to: fluticasone furoate; fluticasone propionate; and mometasone furoate.

In other contemplated embodiments, an active pharmaceutical ingredient may be used in the treatment of tuberculosis or associated symptoms. Contemplated active pharmaceutical ingredients in this regard include, but are not limited to, interferon gamma.

In other contemplated embodiments, an active pharmaceutical ingredient may be used in the treatment of acquired immunodeficiency syndrome (AIDS) or associated symptoms. Contemplated active pharmaceutical ingredients in this regard include, but are not limited to, interferon gamma.

In other contemplated embodiments, an active pharmaceutical ingredient may be used in the treatment of acute myeloid leukemia or associated symptoms. Contemplated active pharmaceutical ingredients in this regard include, but are not limited to, aldesleukin.

In other contemplated embodiments, an active pharmaceutical ingredient may be used in the treatment of amnesia or associated symptoms. Contemplated active pharmaceutical ingredients in this regard include, but are not limited to, midazolam.

In other contemplated embodiments, an active pharmaceutical ingredient may be used in the treatment of amyotrophic lateral sclerosis (ALS) or associated symptoms. Contemplated active pharmaceutical ingredients in this regard include, but are not limited to, aldesleukin.

In other contemplated embodiments, an active pharmaceutical ingredient may be used in the treatment of angina or associated symptoms. Contemplated active pharmaceutical ingredients in this regard include, but are not limited to, etripamil.

In other contemplated embodiments, an active pharmaceutical ingredient may be used in the treatment of anxiety or related conditions. Contemplated active pharmaceutical ingredients in this regard include, but are not limited to: aloradine; and midazolam.

In other contemplated embodiments, an active pharmaceutical ingredient may be used in the treatment of atrial fibrillation or associated symptoms. Contemplated active pharmaceutical ingredients in this regard include, but are not limited to, etripamil.

In other contemplated embodiments, an active pharmaceutical ingredient may be used in the treatment of Buerger's syndrome or associated symptoms. Contemplated active pharmaceutical ingredients in this regard include, but are not limited to, iloprost trometamol.

In other contemplated embodiments, an active pharmaceutical ingredient may be used in the treatment of colorectal cancer or associated symptoms. Contemplated active pharmaceutical ingredients in this regard include, but are not limited to, interferon gamma.

In other contemplated embodiments, an active pharmaceutical ingredient may be used in the treatment of conjunctivitis or associated symptoms. Contemplated active pharmaceutical ingredients in this regard include, but are not limited to: amnion-derived cellular cytokine solution; and azelastine hydrochloride.

In other contemplated embodiments, an active pharmaceutical ingredient may be used in the treatment of critical limb ischemia or associated symptoms. Contemplated active pharmaceutical ingredients in this regard include, but are not limited to, iloprost trometamol.

In other contemplated embodiments, an active pharmaceutical ingredient may be used in the treatment of diabetes insipidus or associated symptoms. Contemplated active pharmaceutical ingredients in this regard include, but are not limited to, desmopressin.

In other contemplated embodiments, an active pharmaceutical ingredient may be used in the treatment of dry eye syndrome or associated symptoms. Contemplated active pharmaceutical ingredients in this regard include, but are not limited to, amnion-derived cellular cytokine solution.

In other contemplated embodiments, an active pharmaceutical ingredient may be used in the treatment of ebola virus infection or associated symptoms. Contemplated active pharmaceutical ingredients in this regard include, but are not limited to, remdesivir.

In other contemplated embodiments, an active pharmaceutical ingredient may be used in the treatment of eczema or associated symptoms. Contemplated active pharmaceutical ingredients in this regard include, but are not limited to, azelastine hydrochloride.

In other contemplated embodiments, an active pharmaceutical ingredient may be used in the treatment of endometriosis or associated symptoms. Contemplated active pharmaceutical ingredients in this regard include, but are not limited to, buserelin.

In other contemplated embodiments, an active pharmaceutical ingredient may be used in the treatment of enuresis or associated symptoms. Contemplated active pharmaceutical ingredients in this regard include, but are not limited to, desmopressin.

In other contemplated embodiments, an active pharmaceutical ingredient may be used in the treatment of epilepsy or associated symptoms. Contemplated active pharmaceutical ingredients in this regard include, but are not limited to: diazepam; and midazolam.

In other contemplated embodiments, an active pharmaceutical ingredient may be used in the treatment of follicular lymphoma or associated symptoms. Contemplated active pharmaceutical ingredients in this regard include, but are not limited to, aldesleukin.

In other contemplated embodiments, an active pharmaceutical ingredient may be used in the treatment of glaucoma or associated symptoms. Contemplated active pharmaceutical ingredients in this regard include, but are not limited to, amnion-derived cellular cytokine solution.

In other contemplated embodiments, an active pharmaceutical ingredient may be used in the treatment of hemophilia A or associated symptoms. Contemplated active pharmaceutical ingredients in this regard include, but are not limited to, desmopressin.

In other contemplated embodiments, an active pharmaceutical ingredient may be used in the treatment of hepatitis B or associated symptoms. Contemplated active pharmaceutical ingredients in this regard include, but are not limited to, interferon gamma.

In other contemplated embodiments, an active pharmaceutical ingredient may be used in the treatment of hepatitis C or associated symptoms. Contemplated active pharmaceutical ingredients in this regard include, but are not limited to, interferon gamma.

In other contemplated embodiments, an active pharmaceutical ingredient may be used in the treatment of immunodeficiency or associated symptoms. Contemplated active pharmaceutical ingredients in this regard include, but are not limited to, aldesleukin.

In other contemplated embodiments, an active pharmaceutical ingredient may include an influenza vaccine. Contemplated influenza vaccines in this regard include, but are not limited to: influenza vaccine; influenza H1N1 vaccine; live attenuated influenza H1N1 vaccine; recombinant influenza H1N1 vaccine; recombinant influenza H3N2 vaccine; recombinant influenza type B vaccine; quadrivalent live attenuated influenza vaccine; trivalent live attenuated influenza vaccine; and cold-adapted trivalent influenza vaccine.

In other contemplated embodiments, an active pharmaceutical ingredient may be used in the treatment of melanoma or associated symptoms. Contemplated active pharmaceutical ingredients in this regard include, but are not limited to, aldesleukin.

In other contemplated embodiments, an active pharmaceutical ingredient may be used in the treatment of menopausal symptoms. Contemplated active pharmaceutical ingredients in this regard include, but are not limited to, estradiol.

In other contemplated embodiments, an active pharmaceutical ingredient may be used in the treatment of mental agitation or associated symptoms. Contemplated active pharmaceutical ingredients in this regard include, but are not limited to, loxapine.

In other contemplated embodiments, an active pharmaceutical ingredient may be used in the treatment of migraine or associated symptoms. Contemplated active pharmaceutical ingredients in this regard include, but are not limited to: dihydroergotamine mesylate; sumatriptan; and vazegepant.

In other contemplated embodiments, an active pharmaceutical ingredient may be used in the treatment of necrotizing enterocolitis or associated symptoms. Contemplated active pharmaceutical ingredients in this regard include, but are not limited to, amnion-derived cellular cytokine solution.

In other contemplated embodiments, an active pharmaceutical ingredient may be used in the treatment of nocturnal polyuria or associated symptoms. Contemplated active pharmaceutical ingredients in this regard include, but are not limited to, desmopressin.

In other contemplated embodiments, an active pharmaceutical ingredient may be used in the treatment of non-Hodgkin's lymphoma or associated symptoms. Contemplated active pharmaceutical ingredients in this regard include, but are not limited to, aldesleukin.

In other contemplated embodiments, an active pharmaceutical ingredient may be used in the treatment of ocular inflammation or associated symptoms. Contemplated active pharmaceutical ingredients in this regard include, but are not limited to, amnion-derived cellular cytokine solution.

In other contemplated embodiments, an active pharmaceutical ingredient may be used in the treatment of oesophagitis or associated symptoms. Contemplated active pharmaceutical ingredients in this regard include, but are not limited to, caspofungin acetate.

In other contemplated embodiments, an active pharmaceutical ingredient may be used in the treatment of osteonecrosis or associated symptoms. Contemplated active pharmaceutical ingredients in this regard include, but are not limited to, iloprost trometamol.

In other contemplated embodiments, an active pharmaceutical ingredient may include a medication for organ transplant rejection. Contemplated medications for treatment of pain in this regard include, but are not limited to, ciclosporin A.

In other contemplated embodiments, an active pharmaceutical ingredient may be used in the treatment of osteoporosis or associated symptoms. Contemplated active pharmaceutical ingredients in this regard include, but are not limited to, calcitonin.

In other contemplated embodiments, an active pharmaceutical ingredient may be used in the treatment of overactive bladder or associated symptoms. Contemplated active pharmaceutical ingredients in this regard include, but are not limited to, desmopressin.

In other contemplated embodiments, an active pharmaceutical ingredient may be used in the treatment of optic neuritis or associated symptoms. Contemplated active pharmaceutical ingredients in this regard include, but are not limited to, amnion-derived cellular cytokine solution.

In other contemplated embodiments, an active pharmaceutical ingredient may be used in the treatment of an otological infection or associated symptoms. Contemplated active pharmaceutical ingredients in this regard include, but are not limited to, immunostimulant ribosomal vaccine.

In other contemplated embodiments, an active pharmaceutical ingredient may be used in the treatment of periodontitis or associated symptoms. Contemplated active pharmaceutical ingredients in this regard include, but are not limited to, amnion-derived cellular cytokine solution.

In other contemplated embodiments, an active pharmaceutical ingredient may be used in the treatment of peripheral vascular disease or associated symptoms. Contemplated active pharmaceutical ingredients in this regard include, but are not limited to, iloprost trometamol.

In other contemplated embodiments, an active pharmaceutical ingredient may be used in the treatment of persistent corneal epithelial defect or associated symptoms. Contemplated active pharmaceutical ingredients in this regard include, but are not limited to, amnion-derived cellular cytokine solution.

In other contemplated embodiments, an active pharmaceutical ingredient may be used in the treatment of prostate cancer or associated symptoms. Contemplated active pharmaceutical ingredients in this regard include, but are not limited to, buserelin.

In other contemplated embodiments, an active pharmaceutical ingredient may be used in the treatment of Raynaud's disease or associated symptoms. Contemplated active pharmaceutical ingredients in this regard include, but are not limited to, iloprost trometamol.

In other contemplated embodiments, an active pharmaceutical ingredient may be used in the treatment of renal cancer or associated symptoms. Contemplated active pharmaceutical ingredients in this regard include, but are not limited to, aldesleukin.

In other contemplated embodiments, an active pharmaceutical ingredient may be used in the treatment of rheumatoid arthritis or associated symptoms. Contemplated active pharmaceutical ingredients in this regard include, but are not limited to, iloprost trometamol.

In other contemplated embodiments, an active pharmaceutical ingredient may be used in the treatment of supraventricular tachycardia or associated symptoms. Contemplated active pharmaceutical ingredients in this regard include, but are not limited to, etripamil.

In other contemplated embodiments, an active pharmaceutical ingredient may be used in the treatment of traumatic brain injury. Contemplated active pharmaceutical ingredients in this regard include, but are not limited to, amnion-derived cellular cytokine solution.

In other contemplated embodiments, an active pharmaceutical ingredient may be used in the treatment of urinary incontinence or associated symptoms. Contemplated active pharmaceutical ingredients in this regard include, but are not limited to, desmopressin.

In other contemplated embodiments, an active pharmaceutical ingredient may be used in the treatment of urticaria or associated symptoms. Contemplated active pharmaceutical ingredients in this regard include, but are not limited to, azelastine hydrochloride.

In other contemplated embodiments, an active pharmaceutical ingredient may be used in the treatment of uterine fibrosis or associated symptoms. Contemplated active pharmaceutical ingredients in this regard include, but are not limited to, buserelin.

In other contemplated embodiments, an active pharmaceutical ingredient may be used in the treatment of viral hemorrhagic fevers or associated symptoms. Contemplated active pharmaceutical ingredients in this regard include, but are not limited to, remdesivir.

In other contemplated embodiments, an active pharmaceutical ingredient may be used in the treatment of von Willebrand's disease or associated symptoms. Contemplated active pharmaceutical ingredients in this regard include, but are not limited to, desmopressin.

Based on the foregoing description, it will be readily understood by those persons skilled in the art that the invention has broad utility and application. Many embodiments and adaptations of the invention other than those specifically described herein, as well as many variations, modifications, and equivalent arrangements, will be apparent from or reasonably suggested by the invention and the foregoing descriptions thereof, without departing from the substance or scope of the invention. Accordingly, while the invention has been described herein in detail in relation to one or more preferred embodiments, it is to be understood that this disclosure is only illustrative and exemplary of the invention and is made merely for the purpose of providing a full and enabling disclosure of the invention. The foregoing disclosure is not intended to be construed to limit the invention or otherwise exclude any such other embodiments, adaptations, variations, modifications or equivalent arrangements, the invention being limited only by the claims appended hereto and the equivalents thereof.

1-28. (canceled)

29. A method of administering an aerosolizable and inhalable pharmaceutical complex to a patient comprising the steps of:
providing a first cartridge housing a first liquid formulation that includes a vasodilator active pharmaceutical ingredient;
providing a second cartridge housing a second liquid formulation that includes a non-vasodilator pharmaceutical ingredient;
aerosolizing the first liquid formulation using an electronic device to facilitate inhalation and pulmonary delivery of the vasodilator active pharmaceutical ingredient, thereby increasing oxygenation in a blood flow of the patient; and
after increasing oxygenation in the blood flow, aerosolizing the second liquid formulation using the electronic device to facilitate inhalation and pulmonary delivery of the non-vasodilator active pharmaceutical ingredient.

30. The method of claim 29, wherein at least one of the first and second liquid formulations is in an aqueous form.

31. The method of claim 29, wherein at least one of the first and second liquid formulations is in a nanoemulsion form.

32-39. (canceled)

40. A method of administering an aerosolizable and inhalable pharmaceutical complex to a patient comprising the steps of:
providing a first cartridge housing a first liquid formulation that includes an active pharmaceutical ingredient that exhibits a first particle size distribution;
providing a second cartridge housing a second liquid formulation that includes an active pharmaceutical ingredient that exhibits a second particle size distribution different than the first particle size distribution;
aerosolizing the first liquid formulation using an electronic device; and
after aerosolizing the first liquid formulation, aerosolizing the second liquid formulation.

41. The method of claim 40, wherein the active pharmaceutical ingredient of the first liquid formulation and the active pharmaceutical ingredient of the second liquid formulation include a common active pharmaceutical ingredient.

42. The method of claim 41, wherein the common active pharmaceutical ingredient includes povidone-iodine.

43. The method of claim 42, wherein the first liquid formulation exhibits a generally turbulent flow upon aerosolization.

44. The method of claim 42, wherein the second liquid formulation exhibits a generally laminar flow upon aerosolization.
